(19) United States
(12) Patent Application Publication
(10) Pub. No.: US 2005/0191365 A1
(43) Pub. Date: Sep. 1, 2005

(54) Antimicrobial food additive and treatment for cooked food, water and wastewater

(76) Inventors: David H. Creasey, Vero Beach, FL (US); Barry W. Cummins, Fort Pierce, FL (US)

Correspondence Address: LAW OFFICES OF BRIAN S STEINBERGER, 101 BREVARD AVENUE, COCOA, FL, 32922, US

(21) Appl. No.: 11/065,678

(22) Filed: Feb. 24, 2005

Related U.S. Application Data
(60) Provisional application No. 60/547,991, filed on Feb. 26, 2004.

(51) Int. Cl. A61K 33/38; A61K 33/34; A61K 33/32; A61K 33/04

(52) U.S. Cl. 424/618; 424/709; 424/637; 424/641

(57) ABSTRACT

A composition of matter with antimicrobial and antibacterial properties for treatment of water, wastewater, processed food and for use as a food additive is provided. The antimicrobial composition inhibits cellular growth of known pathogenic, indicator and spoilage organisms, such as salmonella, stahphylococcus, listeria, E-coli, aerobic and anerobic organisms in wastewater and the like. The antimicrobial composition of the present invention is useful in many situations and conditions in need of disinfectants and sanitizers. One of the primary benefits of the antimicrobial agent is that it inhibits the growth of bacteria that have become antibiotic resistant. In addition, the antimicrobial composition herein does not have any known toxicity to man or the environment.

This invention claims the benefit of priority from U.S. Provisional Application Ser. No. 60/547,991 filed Feb. 26, 2004.

### FIELD OF THE INVENTION

This invention relates to an antimicrobial agent and in particular to a composition of matter, a method of making and using the composition of matter for antimicrobial, anti-bacterial treatment of cooked and processed food, water, wastewater, and for a food additive.

### BACKGROUND AND PRIOR ART

Potable water and wastewater that come from various natural and industrial sources can become contaminated. Water treatment has been an established profession for over 100 years; however, it has only been since the 1970's that national standards for clean water have been established and the public has begun to demand safe drinking water and the clean up of lakes, rivers and streams. There is a need for effective, non-toxic substances to treat potable water and wastewater.

Protein sources in the human food chain, such as, eggs, raw meats, poultry, game birds, milk and dairy products, fish, shrimp, frog legs, and the like, carry the potential for nourishment and the potential for illness and death. Edible vegetation in the human diet, such as fruit, vegetables, and crops harvested and handled in contaminated environments can also carry the potential for illness and death. Well-known pathogens such as salmonella, listeria and E-coli, as well as indicator and spoilage organisms, including staph bacteria can be found prior to and during the processing or harvesting of raw meats, fruit, and vegetables or in partially or fully cooked foodstuffs, animal products and water consumed by humans.

The globalization of business, travel and communication brings increased attention to worldwide exchanges between communities and countries, including the potential globalization of the bacterial ecosystem. Harmful bacteria were once controlled with antibiotics, such as penicillin, in the mid-1940s; but the control no longer exists as more and more antibiotic resistant bacteria appear around the globe. For example, before 1946, about 90 percent of Staphylococcus aureus isolates in hospitals were susceptible to penicillin, by 1952, 75 percent of isolates were penicillin-resistant.

Bacterial resistance to antimicrobial agents has emerged, throughout the world, as one of the major threats both in human and veterinary medicine. Resistance to antibiotics and antimicrobial agents has emerged at an alarming rate because of a variety of factors, such as clustering and overcrowding, the use of antibiotics in animal culture and aquaculture, an increase in the number of elderly people, increased traveling, the sale of antibiotics over the counter, self-treatment with antibiotics, a lack of resources for infection control, and the inappropriate use of antibiotics.

Thus, the world population is at increased risk for acquiring antimicrobial-resistant food and water borne infections. Even a small increase in the prevalence of resistance in the most significant pathogenic bacteria may lead to large increases in the potential for treatment failures and other adverse outcomes, including death.

In addition to pathogenic resistance to antibiotics and antimicrobial agents, it has recently become obvious that trace amounts of chemicals and pharmaceuticals used as antibiotics and anti-bacterial additives are becoming a problem in water treatment plants and waterways. For example, the antimicrobial compound triclocarban has been used widely for decades in hand soaps and other cleaning products and unfavorable environmental characteristics have surfaced. Triclocarban and a related chemical triclosan are among the polychlorinated organic compounds which have been found to have long-term persistence and potential bioaccumulation. The estimated half-life of triclocarban is 1.5 years in aquatic sediments. Thus, anti-microbial and antibacterial substances are becoming toxins in the world's waterways.

Appropriate use of antimicrobial agents in water, food and the environment is necessary to maintain the antimicrobial effectiveness and reduce the potential for the spread of resistant organisms. While therapeutic usage of antimicrobial agents in water and food animals is important to promote animal health and provide an affordable supply of meat, milk, and eggs, it is vital that the long-term effectiveness of antimicrobial agents used in human medicine be preserved. The present invention provides an antimicrobial processing aid and food additive for which there is no known toxicity and no known pathogenic resistance and can be used to protect public health.

In U.S. Pat. Nos. 5,989,595 and 6,242,011 B1 to Cummins, an acidic composition of matter is disclosed that is useful for destroying microorganisms that spoil food, such as fish. The composition of matter, patented by Cummins, is also useful for skin treatment of melanoma and the treatment of other bacteria, and serves as the precursor for the novel antimicrobial agent of the present invention.

The following US Patent publications on the subject matter of the present invention have been identified.

U.S. Pat. No. 5,997,911 to Brinton et al. teach that a simple copper salt, hydroxycarboxylic acid and a buffering agent such as ammonium salts can be solubilized in drinking water for turkeys and swine in an antidiarrheal effective dosage.

U.S. Pat. No. 6,506,737 B1 to Hei et al. disclose an antimicrobial composition for the food industry that may include sulfuric acid, sulfates and an ammonium halide salt to provide a gel-thickened compound for cleaning and sanitizing surfaces among other uses. The use of a halide ingredient limits usage for ingestion by man or animals and would be deleterious to machinery, plants and other vegetation.

U.S. Pat. No. 6,565,893 B1 to Jones et al. describe an aqueous disinfectant for swimming pools and the like, wherein copper sulfate pentahydrate, water, sulfuric acid and ammonium sulfate are combined in a “cold process” requiring that the formulation be maintained at a temperature above 40° F. to keep metallic ions in suspension.

U.S. Patent Pub. No. 2003/0118705 A1 to Cook et al. describe an ingestible disinfectant to eradicate and control pathogens on plants, animals, humans, byproducts of plants and animals and articles infected with pathogens; the disinfectant includes sulfuric acid, water and metallic ions, particularly copper, silver and gold.

Collectively, the above references do not provide a halogen-free composition of matter that is stable under a wide range of temperatures and pH ranges, ingestible, and effective in a wide range of applications, including, but not limited to, treatment of waste water, treatment of drinking water, treatment for seafood, a processing aid for cooked food, and as a food additive. The composition of the present invention is safe and effective in an unlimited number of applications and is also safe for the environment.

### SUMMARY OF THE INVENTION

The first objective of the present invention is to provide a composition of matter and method for its production that inhibits cellular growth of pathogenic organisms.

The second objective of the present invention is to provide a composition of matter and method for its production that inhibits cellular growth of indicator and spoilage organisms.

The third objective of the present invention is to provide a composition of matter and method for its production, for use treatment of waste water.

The fourth objective of the present invention is to provide a composition of matter and method for its production, for use as a food additive.

The fifth objective of the present invention is to provide a composition of matter and method for its production, for the treatment of contaminants on cooked food.

The sixth objective of the present invention is to provide a composition of matter and method for its production, for the treatment of frozen products, such as ice.

The seventh objective of the present invention is to provide a composition of matter and method for its production that is used for treatment of potable water and other beverages.

The eighth objective of the present invention is to provide a composition of matter that inhibits the growth of pathogenic, indicator and spoilage bacteria that have become antibiotic resistant.

A preferred composition of matter with antimicrobial and antibacterial properties for the treatment of water, wastewater, processed food and for use as a food additive, includes sulfuric acid of approximately 94% purity to approximately 99.9% purity, ammonium sulfate, and metallic ions in a uniform suspension within the mixture.

The preferred metallic ions are copper ions, silver ions, zinc ions, magnesium ions and mixtures thereof available from such compounds as, copper sulfate, copper sulfate pentahydrate, copper glutamate, zinc oxide, zinc glutamate, magnesium glutamate, magnesium sulfate, silver oxide and silver sulfate.

The processed mixture of sulfuric acid, ammonium sulfate and metallic ions is further mixed with distilled water to form a solution. The ratio of the distilled water to the total weight of mixture is in a range between approximately 2% to approximately 75% by weight.

The distilled water solution is used in the treatment of facultative waste ponds, treatment of anaerobic wastewater and even the treatment of potable water.

The distilled water solution is used as an antibacterial treatment of cooked food stuff and as an additive to water that is subsequently frozen to form an ice product.

A method for making the antimicrobial composition of matter for use as a food additive, treatment for cooked food, treatment for potable water and wastewater, includes combining sulfuric acid of approximately 94% purity to approximately 99.9% purity in a 1 to 2 volume ratio with distilled water and ammonium sulfate in a ratio of 2.77 pounds of ammonium sulfate per gallon of distilled water to provide a first mixture, then processing the first mixture in a pressurized vessel at a pressure that is above atmospheric pressure and heating the mixture at a temperature in a range between approximately 200 degrees Fahrenheit and approximately 1200 degrees Fahrenheit, for at least 30 minutes, then cooling the mixture, adding a stabilizer which is a portion of the first mixture and comprises 10 weight percent of the total weight of the first mixture, thereby forming a second mixture, adding a compound containing metallic ions to the second mixture to form a third mixture, diluting the third mixture with water; and applying an effective amount of the diluted mixture to foods, water and wastewater.

The preferred metallic ions are copper ions, silver ions, zinc ions, magnesium ions and mixtures thereof available from compounds such as, but not limited to, copper sulfate, copper sulfate pentahydrate, copper glutamate, zinc oxide, zinc glutamate, magnesium glutamate, magnesium sulfate, silver oxide and silver sulfate.

The processing of the first mixture further includes applying direct current (DC) voltage to mixture (I) during the addition of ammonium sulfate; the DC voltage is in a range from approximately 1 amp to approximately 100 amps, more preferably, in a range from approximately 1 amp to approximately 5 amps.

The ratio of the water used to form a solution of the compound containing metallic ions to the total weight of the third mixture is in a range between approximately 2% to approximately 75% by weight.

The preferred antimicrobial composition of the present invention is a processed mixture of metallic salts, sulfuric acid, ammonium sulfate and water that kills undesirable aerobic and anaerobic organisms in facultative wastewater, kills gram negative and gram positive organisms in potable water can be applied to the surface of processed food to control Listeria and pathogenic bacteria, and can be added to water before it is frozen into ice products.

Further objects and advantages of this invention will be apparent from the following detailed description of a presently preferred embodiment, which is illustrated in the accompanying tables and graphs.

### BRIEF DESCRIPTION OF THE FIGURES

FIG. 1 is a graph showing the effect of PHB0020 on pathogenic and spoilage bacterial isolates exposed for 2 minutes.

FIG. 2 is a graph showing the logarithm of reductions in bacterial colony levels.

### DESCRIPTION OF THE PREFERRED EMBODIMENT

Before explaining the disclosed embodiment of the present invention in detail, it is to be understood that the invention is not limited in its application to the details of the particular arrangement shown since the invention is capable of other embodiments. Also, the terminology used herein is for the purpose of description and not of limitation.

It would be useful to discuss the meanings of some words used herein and their applications before discussing the composition of matter and method of using and making the same:

PHB0020—Copper sulfate pentahydrate and/or other forms of copper ions, and silver sulfate and/or other forms of silver ions added to pHarlo for the antimicrobial, anti-bacterial additive of the present invention.

pHarlo—composition of matter claimed in U.S. Pat. Nos. 5,989,595 and 6,242,001 B1 to Cummins and incorporated herein by reference and more completely described below.

Pharlo composition—the processed acidic mixture of sulfuric acid, ammonium sulfate and metallic ions mixed with water to form a solution used in treatments and applications described herein.

E-coli—Escherichia coli, indicator bacteria

Listeria—Listeria monocytogenes, a pathogen

Pseudomonas—Pseudomonas fluorescens, spoilage bacteria

Salmonella—Salmonella typhimurium, a pathogen

Shewanella—Shewanella putrefaciens, spoilage bacteria

Staph—Staphylococcus aureus, a pathogen

The acidic composition of matter and method of making are similar to that described in U.S. Pat. Nos. 5,989,595 and 6,242,011 B1 to Cummins and are incorporated herein by reference.

First, a pressurized vessel is selected that includes a cooling jacket and no electrode attachments; however, the preferred pressurized vessel is fitted with two electrodes, a cathode and anode, to provide a direct current (DC) voltage 1 ft. above the bottom of the container. The electrodes are spaced approximately three (3) feet apart.

The processing steps of the present invention comprise combining sulfuric acid with purity in a range from approximately 94% to approximately 99.9%, in a 1 to 2 volume ratio with distilled water and ammonium sulfate in a ratio of 2.77 pounds of ammonium sulfate per gallon of distilled water to provide mixture (I). The mixture (I) is combined in a pressurized vessel having preferably two strategically placed electrodes, a cathode and anode. During the addition of ammonium sulfate, a direct current (DC) voltage is applied to the mixture. The voltage is applied in a range from approximately one (1) amp to approximately 100 amps, preferably between approximately 1 amp and approximately 5 amps. The mixture is then heated under pressure in a range of from approximately 1 pound per square inch (psi) to approximately 15 psi above atmospheric pressure. Heating of the mixture is in a range of from approximately 2000 Fahrenheit (F) to approximately 1200° F., preferably from approximately 800° F. to approximately 900° F. for approximately 30 minutes. With the application of heat and pressure as specified above, it is understood by persons skilled in the art, that a judicious selection of temperature, time and pressure is required and should be adjusted to maintain a safe chemical reaction.

After cooling the mixture, a stabilizer is added, which comprises approximately 10 weight percent of the total weight of mixture (I). The resulting acidic composition is useful for destroying microorganisms, having a pH of negative 3 (−3). The inventive step of the present invention requires the addition of compounds containing metallic ions for the extensive antimicrobial properties discussed herein. The following physical and chemical properties are observed when undiluted.
pH=−3
stability of metallic ions in solution: from approximately 0 pH up to approximately 9 pH
stability of metallic ions with temperature: from approximately 32° F. to the point of vaporization or approximately 212° F.

Various other compounds with metallic ions may be substituted for copper sulfate pentahydrate. The following metal salts are suitable substitutes:

Copper sulfate, copper glutamate, zinc oxide, zinc glutamate, magnesium glutamate, magnesium sulfate, silver sulfate, silver oxide, and combinations thereof.

Referring now to the composition of pHarlo Blue 0020, hereinafter referred to as PHB0020, it is an antimicrobial, anti-bacterial agent, which has a formulation that is generally recognized as safe (GRAS) by the US Food and Drug Administration. PHB0020 is useful in the pre-harvest and post-harvest treatment of food sources and foods, including, but not limited to, plants, fruit, vegetables, eggs, poultry, seafood, meats, and animal and pork products. The composition is listed below:

| Ingredient | Percentage |
|---|---|
| Copper Sulfate | 16.4 |
| Pentahydrate | |
| Sulfuric Acid | 9.9 |
| (processing aid) | |
| Ammonium | 2.2 |
| sulfate | |
| Distilled water | 71.5 |

The ingredients form a concentrate, which is combined in small amounts of less than 0.10 milliliters (ml) with 1 gallon of water to make PHB0020.

The examples, graphs and charts below provide greater detail on the use and effectiveness of PHB0020 as an antimicrobial agent and food additive.

### EXAMPLE 1

In processing plants for poultry and animal products, it is customary to use various water treatment processes, such as a scalding tank, spray bath, final rinse and chill water tank. The scalding tank is used to dip poultry prior to the removal of feathers; other animals are dipped to remove the outer coating of fur or hair. The scalding process permits cross contamination and spread of pathogens. It is important for the safety of the human food supply to provide an additive that can be used in water treatments to inhibit the growth and spread of pathogens and deleterious bacteria. The ideal additive would not evaporate at boiling point temperatures, would not be destroyed by high temperatures and would not be bound by organic material, such as blood and feces and rendered useless.

The effect of PHB0020 on pathogenic, indicator, and spoilage populations of bacteria associated with broiler chicken carcasses in a poultry scald water application is determined in one embodiment of the present invention.

First, scalder water was collected from the overflow or entrance end of a commercial poultry scalder. The water is sterilized or autoclaved to eliminate all populations of bacteria and bacterial spores to avoid interference during the study. The autoclaved scalder water is evaluated chemically and compared to raw scalder water to ensure that the organic material demand in raw and autoclaved scalder water is similar.

Next, sets of test tubes are prepared by adding 9 milliliters (ml) of sterilized scalder water to sterile polystyrene test tubes. One set is prepared as controls by adding 9 ml of sterilized scalder water to tubes. One set is prepared by adding 9 ml of sterilized scalder water and PHB0020 (the disinfectant) until the pH of 2.2 is achieved.

Each bacterium is exposed, one at a time, to the sterilized scalder water with PHB0020 sanitizer for approximately 2 minutes at approximately 130° F. (55° C.) to mimic scalding.

After the exposure period, one ml of the suspension was enumerated using the aerobic plate count method by pour plating and incubating at approximately 95° F. (35° C.) for 48 hours.

Table I below records microbial growth results in a scalder water project wherein sterilized water was heated to scalding temperatures of in a range of from approximately 120° F. (49° C.) to approximately 140° F. (60° C.), preferably to a temperature of approximately 130° F. (55° C.). Various concentrations of PHB0020 are added in a range between approximately 0.4 parts per million (ppm) to approximately 0.8 ppm, preferably at approximately 0.6 ppm and colonies of pathogens, indicator bacteria and spoilage bacteria are exposed to the treated scalder water.

TABLE I
Scalder Water Project

| | | | |
|---|---|---|---|
| Control | Colonies forming | Log of | Growth after Exposure |
| Sample No.: | Bacteria Units | Reduction | to Treated Scalder Water |

Bacteria: Salmonella typhimurium

| | | | |
|---|---|---|---|
| 1 | 430 | 2.633468 | negative (no growth) |
| 2 | 880 | 2.944483 | negative |
| 3 | 970 | 2.986772 | negative |
| 4 | 450 | 2.653213 | negative |
| 5 | 620 | 2.792392 | negative |
| 6 | 700 | 2.845098 | negative |
| 7 | 1140 | 3.056905 | negative |
| 8 | 620 | 2.792392 | negative |
| 9 | 580 | 2.763428 | negative |

Bacteria: Staphylococcus aureus

| | | | |
|---|---|---|---|
| 1 | 530 | 2.724276 | negative (no growth) |
| 2 | 550 | 2.740363 | one (1) colony growing |
| 3 | 580 | 2.763428 | negative |
| 4 | 500 | 2.698970 | negative |
| 5 | 540 | 2.732394 | negative |
| 6 | 420 | 2.623249 | negative |
| 7 | 530 | 2.724276 | negative |
| 8 | 480 | 2.681241 | one (1) colony growing |
| 9 | 470 | 2.672098 | negative |

Bacteria: Pseudomonas fluorescens

| | | | |
|---|---|---|---|
| 1 | 540 | 2.73234 | negative |
| 2 | 880 | 2.944483 | negative |
| 3 | 790 | 2.897627 | negative |
| 4 | 620 | 2.792392 | negative |
| 5 | 1120 | 3.049218 | negative |
| 6 | 790 | 2.897627 | one (1) colony growing |
| 7 | 5200 | 3.716003 | negative |
| 8 | 1360 | 3.133539 | negative |
| 9 | 1040 | 3.017033 | negative |

Bacteria: Listeria monocytogenes

| | | | |
|---|---|---|---|
| 1 | 1720 | 3.235528 | five (5) colonies growing |
| 2 | 1840 | 3.264818 | six (6) colonies growing |
| 3 | 1440 | 3.158362 | negative (no growth) |
| 4 | 1820 | 3.260071 | five (5) colonies growing |
| 5 | 1440 | 3.158362 | one (1) colony growing |
| 6 | 1880 | 3.274158 | negative |
| 7 | 1720 | 3.235528 | negative |
| 8 | 1720 | 3.235528 | negative |
| 9 | 1740 | 3.240549 | negative |

Bacteria: Shewanella putrefaciens

| | | | |
|---|---|---|---|
| 1 | 50 | 1.698970 | negative (no growth) |
| 2 | 50 | 1.698970 | negative |
| 3 | 60 | 1.778151 | negative |
| 4 | 20 | 1.301030 | negative |
| 5 | 50 | 1.698970 | negative |
| 6 | 70 | 1.845098 | negative |
| 7 | 80 | 1.903090 | negative |
| 8 | 20 | 1.301030 | negative |
| 9 | 30 | 1.477121 | negative |

Bacteria: Escherichia coli

| | | | |
|---|---|---|---|
| 1 | 15100000 | 7.178977 | 460 colonies growing |
| 2 | 12900000 | 7.110590 | negative (no growth) |
| 3 | 13300000 | 7.123852 | 32 colonies growing |
| 4 | 12200000 | 7.086360 | 1170 colonies growing |
| 5 | 13400000 | 7.127105 | 4700 colonies growing |
| 6 | 12200000 | 7.086360 | 57 colonies growing |
| 7 | 14200000 | 7.152288 | 900 colonies growing |
| 8 | 13600000 | 7.133539 | 410 colonies growing |
| 9 | 7600000 | 6.880814 | 37 colonies growing |

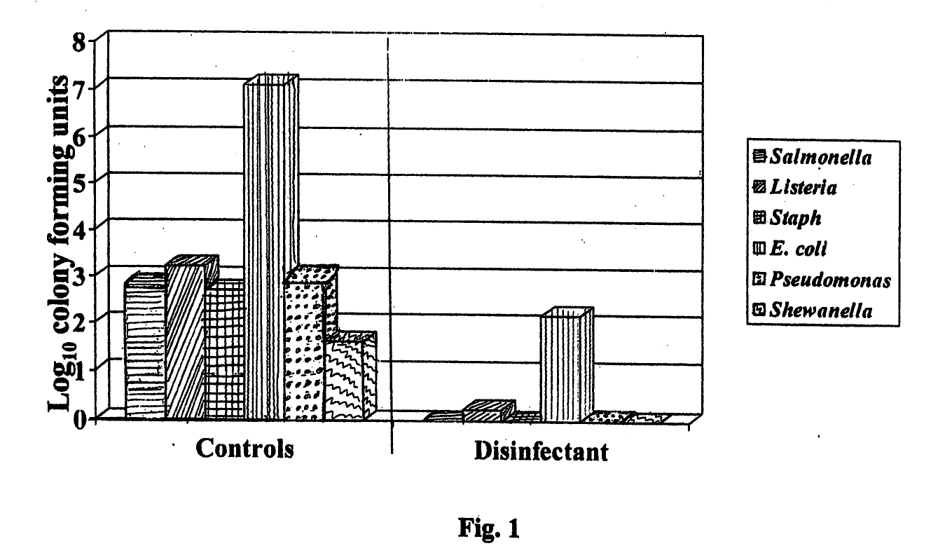

Referring now to FIG. 1, the graph shows the effect of PHB0020 on pathogenic and spoilage bacteria identified in the table above. The graph is divided in two sections, on the left is the control showing the logarithm of colony forming units for each bacterium and on the right is the graph of colony forming units after each bacterium is exposed for 2 minutes to scalder water treated with PHB0020. The graph shows that Listeria, a gram-positive bacterium, is hard to kill and E coli, a very prolific bacterium, has the highest reduction after a 2 minute exposure.

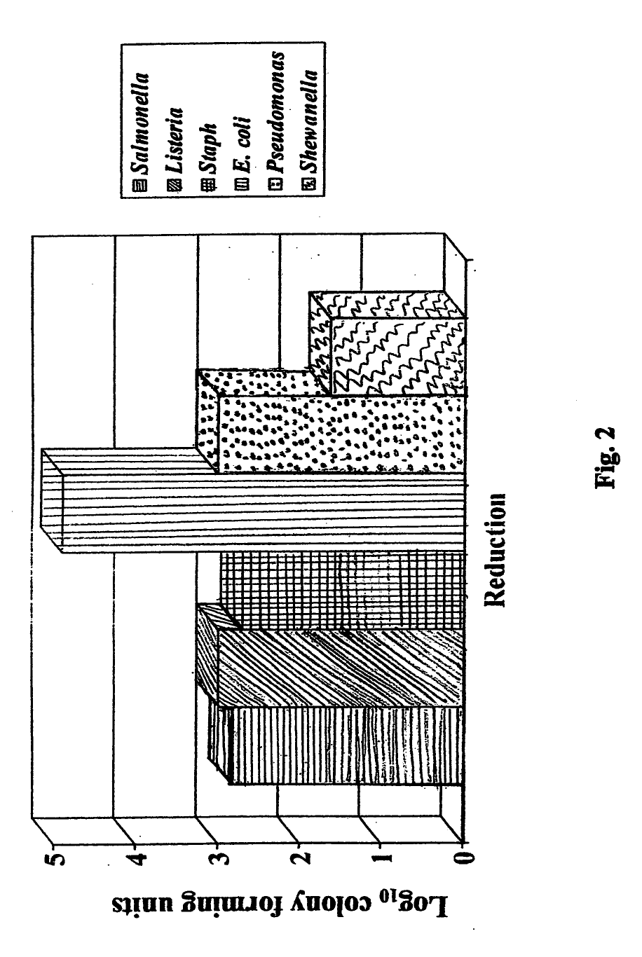

In FIG. 2, the graph shows the logarithm of the reduction of bacterial levels for each bacterium. In most cases the log of colony forming units is less than three, with the most prolific bacterium, E coli having a log of less than five.

Thus, PHB0020 functions as an antimicrobial agent, disinfectant, or sanitizer and is extremely effective for eliminating populations of pathogenic, indicator and spoilage bacteria in commercial scalder water under industrial scalding conditions. PHB0020 is n effective means for controlling bacteria in scalder water and may be used for controlling cross-contamination during scalding. Disinfection of poultry scalder water is crucial because it is the first area within the plant in which birds are immersed in a common bath and bacteria may be transferred from bird to bird.

The efficacy of PHB0020 as an antimicrobial agent is suitable for many other uses and in the quantitative ranges identified in Table II below:

TABLE II

Use Levels in Milligrams
per Liter (mg/l):

| Application for PHB0020: | Range | Target |
|---|---|---|
| Potable water | 0.4 to 1.5 mg/l | 1.0 mg/l |
| Wastewater | 0.6 to 1.0 mg/l | 0.8 mg/l |
| Contaminants on cooked | 0.4 to 1.0 mg/l | 0.8 mg/l |
| food | | |
| Preservative coating | 0.4 to 0.8 mg/l | 0.6 mg/l |
| Ingredient in Ice Products | 0.6 to 1.0 mg/l | 0.8 mg/l |

The table above identifies some of the applications for the present invention; it is an indication of the enormous commercial potential for the novel antimicrobial composition that can be used to protect public health.

The composition can be produced in several forms when diluted with distilled water, such as, an aerosol, mist, vapor or fog to produce micron sized particles that remain in suspension in the air for a period of time and act on airborne pathogens that come in contact with the composition.

### EXAMPLE 2

### Wastewater Treatment

A Pharlo composition containing 0.002% PHB0020 can be added to facultative waste pond areas containing both aerobic and anaerobic organisms and to wastewater with volatile organic sulfur compounds that give off a very noxious sulfur odor. The Pharlo composition is added in a range between approximately 0.6 to approximately 1.0 milligrams (mg) per liter (1); preferably at a concentration of approximately 0.8 mg/l.

The Pharlo composition can also be used in the same concentration range during the disinfection step of a wastewater treatment plant of other wastewater handling facility.

The Pharlo composition reacts with organic materials in the wastewater, thereby controlling the growth of facultative waste organisms and anaerobic organisms. Thus, a substantially increased volume and variety of sewage or wastewater can be treated while avoiding draw backs commonly associated with entirely anaerobic treatments or entirely aerobic treatments.

When the Pharlo composition is added, the anaerobic organisms take in sulfur as a food source. Hydrogen ions and copper ions are also present in large amounts and the anaerobic organism fights to get rid of the hydrogen and has to produce large quantities of an enzyme, adenosine diphosphate (ADP) to expel hydrogen ions; this action lowers the pH inside the organism resulting in the death of the organism Simultaneously, the aerobic organisms in the wastewater are unable to grow and reproduce because the metallic ions in the Pharlo composition interfere with electron receptors that usually stimulate the growth of the aerobic organisms.

A person skilled in the art can easily adjust the concentrations of the Pharlo composition to achieve the desired results of controlling the growth and reproduction of noxious, foul-smelling organisms in wastewater.

### EXAMPLE 3

### Potable Water Treatment

A Pharlo composition containing 0.002% PHB0020 is added to a drinking water supply. The concentration of the Pharlo composition is in a range between approximately 0.05 mg/l and approximately 1.5 mg/l; preferably at a concentration of approximately 1.0 mg/l. The formulation is similar to that used in wastewater treatments.

The Pharlo composition for the treatment of potable water kills gram negative and gram positive organisms, such as E-coli and listeria and kills bacterium that produce endotoxins.

### EXAMPLE 4

### Food Additive

A Pharlo composition containing 0.005% PHB0020 is added to water before it is frozen into ice products used, for example, during seafood processing. The ice product can be used in a shrimp hold or fish hold to prolong the quality and shelf-life of the seafood. The Pharlo composition is added to water in a concentration range between approximately 0.6 to approximately 1.0 milligrams (mg) per liter (1); preferably at a concentration of approximately 0.8 mg/l. Subsequently, the water containing the Pharlo composition is frozen into cubes or chips of ice.

The Pharlo composition can also be added to other foods and beverages as a processing aid.

### EXAMPLE 5

### Cooked or Processed Food

A Pharlo composition containing approximately 0.05% PHB0020 and approximately 99.95% water is used as a topical preservative on cooked or processed foods, such as chicken breast, bologna, cooked pork, and the like. The Pharlo composition can have a concentration in a range between approximately 0.4 milligrams (mg)/liter (1) to approximately 1.0 milligrams (mg) per liter (1); preferably at a concentration of between approximately 0.6 mg/l to approximately 0.8 mg/l for this application.

The Pharlo composition is sprayed on the surface of the cooked or processed food or it can be electrostatically applied using a spraying device that is electrically charged. This application is used to control Listeria and any pathogenic bacteria that may still be present after processing.

The use and effectiveness of a Pharlo composition of the present invention have been demonstrated and disclosed herein for improving the quality and health of our environment.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the breadth and scope of the claims here appended.

1. A composition of matter with antimicrobial and antibacterial properties for the treatment of water, wastewater, processed food and use as a food additive, comprising:
sulfuric acid of approximately 94% purity to approximately 99.9% purity;
ammonium sulfate; and
metallic ions in a uniform suspension within the mixture.

2. The composition of claim 1, wherein the metallic ions are selected from the group consisting of copper ions, silver ions, zinc ions, magnesium ions and mixtures thereof.

3. The composition of claim 1, wherein the compound containing metallic ions is selected from at least one of: copper sulfate, copper sulfate pentahydrate, copper glutamate, zinc oxide, zinc glutamate, magnesium glutamate, magnesium sulfate, silver oxide and silver sulfate.

4. The composition of claim 1, that is further mixed with distilled water to form a solution.

5. The composition of claim 4, wherein the ratio of the distilled water to the total weight of mixture is in a range between approximately 2% to approximately 75% by weight.

6. The composition of claim 5, wherein the distilled water solution is used in the treatment of facultative waste ponds.

7. The composition of claim 5, wherein the distilled water solution is used to treat anaerobic wastewater.

8. The composition of claim 5, wherein the distilled water solution is used to treat potable water.

9. The composition of claim 1 that is used as an antibacterial treatment of cooked food stuff.

10. The composition of matter of claim 1 that is used as an additive to water that is subsequently frozen to form an ice product.

11. An antimicrobial composition of matter for use as a food additive, treatment for cooked food, treatment for potable water and wastewater, made by the process of:
(a) combining sulfuric acid of approximately 94% purity to approximately 99.9% purity in a 1 to 2 volume ratio with distilled water and ammonium sulfate in a ratio of 2.77 pounds of ammonium sulfate per gallon of distilled water to provide mixture (I);
(b) processing the mixture (I) in a pressurized vessel at a pressure that is above atmospheric pressure and heating the mixture at a temperature in a range between approximately 200 degrees Fahrenheit and approximately 1200 degrees Fahrenheit, for at least 30 minutes;
(c) cooling the mixture;
(d) adding a stabilizer which is a portion of mixture (I) and comprises 10 weight percent of the total weight of mixture (I), thereby forming mixture (II);
(e) adding a compound containing metallic ions to mixture (II) to form mixture (III);
(f) diluting mixture (III) with water; and
(g) applying an effective amount of the diluted mixture to foods, water and wastewater.

12. The antimicrobial composition made by the process of claim 11, wherein the metallic ions are selected from the group consisting of copper ions, silver ions, zinc ions, magnesium ions and mixtures thereof.

13. The antimicrobial composition made by the process of claim 11, wherein the compound containing metallic ions is selected from at least one of: copper sulfate, copper sulfate pentahydrate, copper glutamate, zinc oxide, zinc glutamate, magnesium glutamate, magnesium sulfate, silver oxide and silver sulfate.

14. The antimicrobial composition made by the process of claim 11, further comprising the step of applying direct current (DC) voltage to mixture (I) during the addition of ammonium sulfate.

15. The antimicrobial composition made by the process of claim 14, wherein the DC voltage is in a range from approximately 1 amp to approximately 100 amps.

16. The antimicrobial composition made by the process of claim 15, wherein the DC voltage is in a range from approximately 1 amp to approximately 5 amps.

17. The antimicrobial composition made by the process of claim 11, wherein the ratio of the water used to form a solution of the compound containing metallic ions to the total weight of mixture (III) is in a range between approximately 2% to approximately 75% by weight.

18. An antimicrobial composition comprising a processed mixture of metallic salts, sulfuric acid, ammonium sulfate and water that kills undesirable aerobic and anaerobic organisms in facultative wastewater.

19. An antimicrobial composition comprising a processed mixture of metallic salts, sulfuric acid, ammonium sulfate and water that kills gram negative and gram positive organisms in potable water.

20. An antimicrobial composition comprising a processed mixture of metallic salts, sulfuric acid, ammonium sulfate and water that is applied to the surface of processed food to control Listeria and pathogenic bacteria.

21. An antimicrobial composition comprising a processed mixture of metallic salts, sulfuric acid, ammonium sulfate and water that is added to water before it is frozen into ice products.
